(19) United States
(12) Patent Application Publication
(10) Pub. No.: US 2025/0252758 A1
(43) Pub. Date: Aug. 7, 2025

(54) DETERMINING PREDICTED OUTCOMES OF SUBJECTS WITH CANCER BASED ON SEGMENTATIONS OF BIOMEDICAL IMAGES

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: David Joon HO, New York, NY (US); Chad VANDERBILT, New York, NY (US); Narasimhan P. AGARAM, New York, NY (US); Thomas J. FUCHS, New York, NY (US); Meera R. HAMEED, New York, NY (US)

(21) Appl. No.: 18/881,208

(22) PCT Filed: Jul. 5, 2023
(86) PCT No.: PCT/US2023/069620

§ 371 Date: Jan. 3, 2025

Related U.S. Application Data
(60) Provisional application No. 63/358,644, filed on Jul. 6, 2022.

(51) Int. Cl. G06V 20/69; G06T 7/00; G06V 10/25; G06V 10/82

(52) U.S. Cl. CPC G06V 20/69; G06T 7/0012; G06V 10/25; G06V 10/82; G06T 2207/10056; G06T 2207/20081; G06T 2207/20084; G06T 2207/30024; G06T 2207/30096

(57) ABSTRACT

Presented herein are systems and methods of determining predicted outcomes of subjects with cancer from biomedical images. A computing system may identify a biomedical image of a tissue sample from a subject with cancer. The biomedical image may have (i) a first region of interest (ROI) corresponding to viable tumor and (ii) a second ROI corresponding to necrotic tumor in the tissue sample. The computing system may apply a machine learning model to the biomedical image to determine (i) a first segment identifying the first ROI and (ii) a second segment identifying the second ROI. The computing system may determine a ratio between a first size of the first segment associated with the viable tumor and a second size of the second segment associated with the necrotic tumor. The computing system may generate a value indicative of a predicted outcome of the cancer in the subject using the ratio.

### CROSS-REFERENCE TO RELATED PATENT APPLICATIONS

This application claims priority from Provisional Application U.S. Application 63/358,644, filed Jul. 6, 2022, titled “Deep Learning-Based Objective and Reproducible Osteosarcoma Chemotherapy Response Assessment and Outcome Prediction,” which is incorporated herein by reference in its entirety.

### BACKGROUND

A computing device may apply one or more computing vision techniques on a digital image to generate outputs. The outputs may include various information about the image.

### SUMMARY

Aspects of the present disclosure are directed to systems, methods, and computer-readable media for determining predicted outcomes of subjects with cancer from biomedical images. A computing system may identify a biomedical image of a tissue sample from a subject with cancer. The biomedical image may have (i) a first region of interest (ROI) corresponding to viable tumor in the tissue sample and (ii) a second ROI corresponding to necrotic tumor in the tissue sample. The computing system may apply a machine learning model to the biomedical image to determine (i) a first segment identifying the first ROI in the biomedical image and (ii) a second segment identifying the second ROI in the biomedical image. The computing system may determine a ratio between a first size of the first segment associated with the viable tumor and a second size of the second segment associated with the necrotic tumor. The computing system may generate a value indicative of a predicted outcome of the cancer in the subject using the ratio. The computing system may store, using one or more data structures, an association between the subject and the value indicative of the predicted outcome.

In some embodiments, the computing system may classify the subject into a risk stratification category of a plurality of risk stratification categories based on a comparison of the value with a threshold. In some embodiments, the computing system may maintain a measure of progression of the cancer in the subject using the risk stratification category of the subject over a plurality of time instances.

In some embodiments, the computing system may determine the threshold to compare against, based on a plurality of values each indicative of predicted outcome determined for a respective subject of a plurality of subjects. In some embodiments, the computing system may generate the value indicating at least one of an overall survival or a progression-free survival of the subject, based on the ratio of the first size of the first segment associated with the viable tumor and the second size of the second segment associated with the necrotic tumor.

In some embodiments, the computing system may receive a corresponding plurality of biomedical images of a respective plurality of tissue samples obtained from an anatomical site for the cancer of the subject over a corresponding plurality of time instances. In some embodiments, the computing system may generate the value indicative of the predicted outcome of the cancer for the subject at a respective time instance of the plurality of time instances at which the tissue sample was obtained.

In some embodiments, the computing system may receive, via an imaging acquirer, a plurality of biomedical images each corresponding to a whole slide image (WSI) of a respective tissue sample stained to differentiate the viable tumor and the necrotic tumor from a remainder of the tissue sample. In some embodiments, the computing system may determine the ratio between (i) a respective first size of the first segment associated with the viable tumor and (ii) a respective second size of the second segment associated with the necrotic tumor determined from each of the plurality of biomedical images.

In some embodiments, the computing system may apply the machine learning model to the biomedical image to determine a plurality of segments. Each of the plurality of segments may correspond to a respective morphological classification of a plurality of morphological classifications for the tissue sample. In some embodiments, the first size identifies a first number of pixels of the first segment associated with the viable tumor, and wherein the second size identifies a second number of pixels of the second segment associated with the necrotic tumor.

In some embodiments, the machine learning model may be established using a training dataset comprising a plurality of examples. Each of the plurality of examples may identify (i) a respective second biomedical image of a second tissue sample having (a) a third ROI corresponding to viable tumor in the second tissue sample and (b) a fourth ROI corresponding to necrotic tumor in the second tissue and (ii) an annotation identifying the third ROI and the fourth ROI in the respective second biomedical image. In some embodiments, the computing system may provide information to define a treatment to administer to the cancer in the subject based on the association between the value and the subject, wherein the cancer includes one of bone cancer, lung cancer, breast cancer, or colon cancer.

### BRIEF DESCRIPTION OF THE DRAWINGS

FIG. 1 is a block diagram of the proposed method. Top: Currently, an osteosarcoma case with multiple slides is assessed via a microscope to estimate necrosis ratio and to predict outcome. Bottom: Deep learning-based segmentation by Deep Multi-Magnification Network was used to segment multiple tissue subtypes, to count the number of pixels for viable tumor (VT) and necrotic tumor (NT), to estimate necrosis ratio, and to predict outcome.

FIG. 2 is an osteosarcoma data set containing 103 cases with 3134 whole slide images (WSIs). Fifteen cases were used to train the segmentation model, and the other 88 cases were used to test the model. More specifically, 80 cases were used to evaluate necrosis ratio assessment, 75 cases were used to predict overall survival (OS), and 64 cases were used to predict progression-free survival (PFS).

FIGS. 3A-D shows a Multiclass segmentation of two osteosarcoma whole slide images. Whole slide images (A and C) and their segmentation predictions (B and D). Viable tumor is segmented in red, necrosis/nonviable bone is segmented in blue, necrosis/fibrosis without bone is segmented in yellow, normal bone is segmented in green, normal tissue is segmented in orange, normal cartilage is segmented in brown, and blank is segmented in gray. Scale bar=5 mm (A and C).

FIGS. 4A-F shows a Segmentation of viable tumor (A and B), necrosis/nonviable bone (C and D), and necrosis/fibrosis without bone (E and F). Viable tumor is segmented in red, necrosis/nonviable bone is segmented in blue, and necrosis/fibrosis without bone is segmented in yellow. Scale bars: 100 m (A); 200 m (C and E).

FIG. 5 shows an outcome prediction. A: Patient stratification based on overall survival (OS) outcome at the conventional 90% cutoff threshold from manually assessed pathology reports, achieving P=0.045. B: Patient stratification based on OS outcome at the same 90% cutoff threshold from the deep learning model, achieving P=0.0031. The deep learning model performed a better stratification than manual assessment of glass slides. C: Patient stratification based on OS outcome at the 80% cutoff threshold from the deep learning model, achieving P=2.4×10-6. The cutoff threshold for the deep learning model and the data set can be tuned to have better stratification because of its objective and reproducible manner. D: Patient stratification based on progression-free survival outcome at the 60% cutoff threshold from the deep learning model, achieving P=0.016.

FIG. 6 depicts a block diagram of a system for determining predicted outcomes of subjects with cancer from biomedical images, in accordance with an illustrative embodiment;

FIG. 7A depicts a block diagram of a process for segmenting biomedical images in the system for determining predicted outcomes, in accordance with an illustrative embodiment;

FIG. 7B depicts a block diagram of a process for providing information on predicted outcomes in the system for determining predicted outcomes, in accordance with an illustrative embodiment;

FIG. 8 depicts a flow diagram of a method determining predicted outcomes of subjects with cancer from biomedical images, in accordance with an illustrative embodiment.

FIG. 9 depicts a block diagram of a server system and a client computer system in accordance with an illustrative embodiment.

### DETAILED DESCRIPTION

Following below are more detailed descriptions of various concepts related to, and embodiments of, systems and methods for determining predicted outcomes of subjects with cancer from biomedical images. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

Section A describes deep learning-based objective and reproducible osteosarcoma chemotherapy response assessment and outcome prediction.

Section B describes systems and methods for determining predicted outcomes of subjects with cancer from biomedical images.

Section C describes a network environment and computing environment which may be useful for practicing various computing related embodiments described herein.

### A. Deep Learning-Based Objective and Reproducible Osteosarcoma Chemotherapy Response Assessment and Outcome Prediction

Osteosarcoma is the most common primary bone cancer, whose standard treatment includes pre-operative chemotherapy followed by resection. Chemotherapy response is used for prognosis and management of patients. Necrosis is routinely assessed after chemotherapy from histology slides on resection specimens, where necrosis ratio is defined as the ratio of necrotic tumor/overall tumor. Patients with necrosis ratio ≥90% are known to have a better outcome. Manual microscopic review of necrosis ratio from multiple glass slides is semiquantitative and can have intraobserver and interobserver variability. In this study, an objective and reproducible deep learning-based approach was proposed to estimate necrosis ratio with outcome prediction from scanned hematoxylin and eosin whole slide images (WSIs). To conduct the study, 103 osteosarcoma cases with 3134 WSIs were collected. Deep Multi-Magnification Network was trained to segment multiple tissue subtypes, including viable tumor and necrotic tumor at a pixel level and to calculate case-level necrosis ratio from multiple WSIs. Necrosis ratio estimated by the segmentation model highly correlates with necrosis ratio from pathology reports manually assessed by experts. Furthermore, patients were successfully stratified to predict overall survival with P=2.4×10⁻⁶ and progression-free survival with P=0.016. This study indicates that deep learning can support pathologists as an objective tool to analyze osteosarcoma from histology for assessing treatment response and predicting patient outcome.

Osteosarcoma is the most common primary bone cancer, with an incidence of 4 to 5 cases per million worldwide per year. Induction chemotherapy before surgery is the standard of care for patients with osteosarcoma. Multiple studies have shown that necrosis ratio, defined as ratio of necrotic tumor/overall tumor, from histologic assessments of resected samples, is one of the important prognostic factors that correlates with patient outcome. Treatment response to chemotherapy includes necrosis, fibrosis, and/or hyalinization; and necrosis ratio estimation by pathologists includes all three pathologic features. The 5-year overall survival (OS) rate for patients whose necrosis ratio is ≥90% is approximately 80%. However, manually assessing tumor necrosis from multiple hematoxylin and eosin (H&E)-stained slides is semiquantitative and is prone to interobserver and intraobserver variability. Necrosis ratio estimation of osteosarcoma on an H&E-sectioned slide at different time points has been shown to have interclass correlation coefficient of 0.652 between six pathologists.

Deep learning, a subfield of machine learning, has been widely studied for the analysis of whole slide images (WSIs) because of its objective and reproducible nature. Multiple groups have developed deep learning models for osteosarcoma that can segment viable tumor and necrotic tumor. Although these models achieve acceptable performance, neither comparison with manually assessed necrosis ratio nor correlation with patient outcome data has been performed.

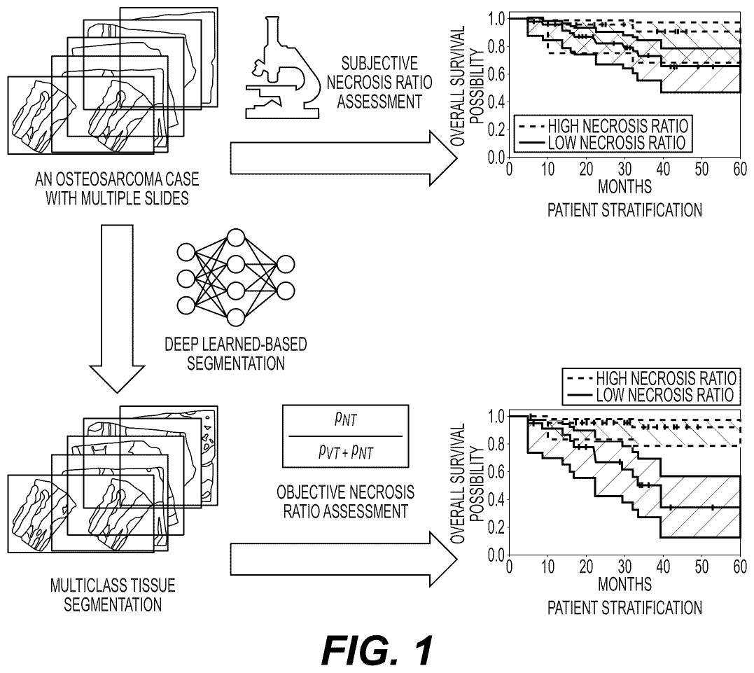

In the present disclosure, a complete pipeline that segments multiple tissue subtypes, including viable tumor and necrotic tumor, at a pixel level from multiple WSIs was proposed to estimate case-level necrosis ratio in an objective and reproducible manner. The estimated necrosis ratio was then correlated with OS and progression-free survival (PFS) outcome data. FIG. 1 shows the block diagram of the proposed method. For pixel-wise segmentation, Deep Multi-Magnification Network was used to accurately segment multiple tissue subtypes. Case-level necrosis ratio can be calculated from segmentation predictions of multiple WSIs by counting the number of pixels of viable tumor and necrotic tumor on WSIs. These data were used to correlate OS and PFS. In addition, the cutoff threshold was tuned to stratify patients specifically for this segmentation model and this data set. The technical details of the method and proof of concept have been previously published at a machine learning conference. The method may be extended to the largest known cohort of digital slide images from patients with osteosarcoma. The main aims of the study were as follows: i) to collect the largest osteosarcoma data set, ii) to develop and release a pixel-wise osteosarcoma segmentation model, iii) to estimate case-level necrosis ratio and compare with manually assessed ratio from pathologists, and iv) to correlate necrosis ratio with the OS and PFS outcome data.

### 1. Materials and Methods

### Data Set

This study was approved by the Institutional Review Board at Memorial Sloan Kettering Cancer Center (protocol number 18-013). After Institutional Review Board approval, osteosarcoma cases with resection materials available at Memorial Sloan Kettering Cancer Center were selected. The resection cases were selected from 2002 to 2020. All cases had preoperative chemotherapy followed by resection. Detailed treatment information was available on 84 cases; and the patients received combination chemotherapy, including cisplatin, doxorubicin, high-dose methotrexate, and/or etoposide or ifosfamide. The resected specimens are routinely sliced along the long axis, and one to three representative slabs are mapped and labeled as per anatomic orientation. After routine processing, the H&E-stained slides are examined microscopically for necrosis assessment (necrotic tumor divided by overall tumor). The pathology reports were reviewed, and the documented percentages of therapy-related changes were recorded. Whenever available, the follow-up data were retrieved from the clinical database. During the previous study, 55 cases with 1578 WSIs were collected. To increase the data set, 48 additional cases with 1556 WSIs digitized in ×20 magnification by Aperio AT2 (Leica Biosystems, Buffalo Grove, IL) scanners at Memorial Sloan Kettering Cancer Center were collected. In total, the data set contains 103 cases with 3134 WSIs, where mean and median of the number of WSIs per case are 30.4 and 27, respectively.

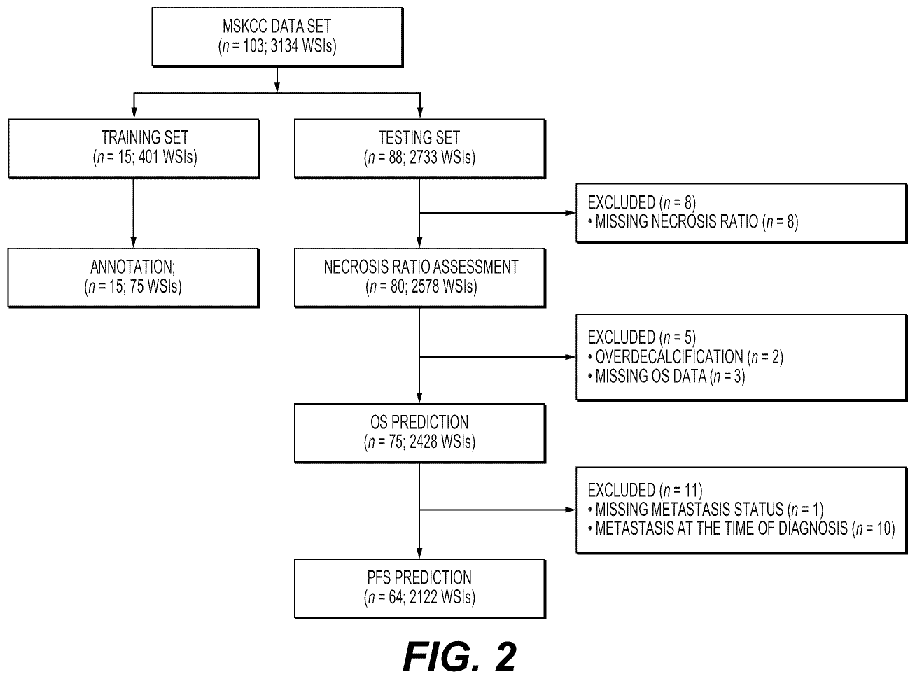

To train the pixel-wise tissue segmentation model, two pathologists annotated tissue regions with concordance to avoid variability on 75 WSIs from 15 training cases. The training cases were selected on the basis of heterogeneous percentage of necrosis and the distribution of seven classes (viable tumor, necrosis/nonviable bone, necrosis/fibrosis without bone, normal bone, normal tissue, normal cartilage, and blank). The two pathologists annotated distinctive morphologic patterns of the seven classes on a subset of WSIs from the training cases, which was sufficient for the model to learn the patterns. The remaining 88 cases were used to test the segmentation model. Because pathologists microscopically review all glass slides to assess necrosis ratio, all WSIs on testing cases were utilized to calculate necrosis ratio. First, 80 cases were used to evaluate necrosis ratio estimation after excluding eight cases missing necrosis ratio in pathology reports. Next, 75 cases were used to predict OS after excluding two cases with overdecalcification and three cases missing OS outcome data. Last, 64 cases were used to predict PFS after excluding one case missing metastasis status and 10 cases who presented with metastases at the time of diagnosis. FIG. 2 shows a Consolidated Standards of Reporting Trials (CONSORT) flow diagram of the data set.

### Tissue Segmentation

Case-level necrosis ratio consists of the ratio of the area of necrotic tumor/the area of overall tumor on a set of osteo-sarcoma slides. Therefore, accurate pixel-wise segmentation would be necessary to count the number of pixels for viable tumor and the number of pixels for necrotic tumor on a set of osteosarcoma WSIs and to estimate the case-level necrosis ratio. WSIs are made up of giga-pixels that cannot be processed as one image because of their large size. Instead, they need to be processed in patches, which are cropped square-shaped regions from the WSIs. In this study, the Deep Multi-Magnification Network, which processes a set of patches in size of 256×256 pixels in ×20, ×10, and ×5 magnifications centered at the same coordinate, was used to accurately generate pixel-wise tissue segmentation predictions of a patch in size of 256×256 pixels in ×20 magnification.

To train the segmentation model, Deep Interactive Learning was used to efficiently annotate a limited set of osteosarcoma training cases. Deep Interactive Learning applies an iterative approach of correcting (or annotating) mislabeled regions from a previous model and fine-tuning the model with the additionally corrected patches to a training set. In this study, the model segmenting seven classes, including viable tumor, necrosis/nonviable bone, necrosis/fibrosis without bone, normal bone, normal tissue, normal cartilage, and blank, was fine-tuned. Specifically, regions with treatment effect with an increased density of inflammatory cells, macrophages, and stromal cells were found to be incorrectly labeled as viable tumor by the previous segmentation model. To fine-tune the model with these morphologic patterns, two additional cases with 26 WSIs containing these patterns were included in the training set. Without any additional manual annotation, these mislabeled regions from the two cases were extracted in patches with the corresponding correct labels (necrosis/fibrosis without bone). For optimization, weighted cross entropy was used as the loss function with stochastic gradient descent, with a learning rate of 5×10⁻⁶, a momentum of 0.99, and a weight decay of 10⁻⁴ for 10 epochs. The final model was selected on the basis of the highest mean intersection over union on the validation set, which is a subset of the training set not used for optimization.

Because giga-pixel WSIs are too large to be segmented at once, patches were segmented starting from a window at the top, left corner of the WSIs and sliding the window to horizontal and vertical directions by 256 pixels until the entire WSIs are segmented. Otsu Algorithm was not used because some necrosis regions can be excluded because of their pixel intensities. All of the implementation for training and inference was done on PyTorch software version 1.3.1, and all experiments were conducted on a Tesla V100 GPU (Nvidia, Santa Clara, CA). WSIs and their segmentation predictions were visualized by the Memorial Sloan Kettering Cancer Center slide viewer.

After all the WSIs in a case are segmented, a case-level necrosis ratio from multiple WSIs, estimated by the deep learning model, r_{DL}, is calculated as follows:

$${r}_{DL} = \frac{{p}_{NT}}{{p}_{VT} + {p}_{NT}} \qquad ( 1 )$$

where p_{VT} and p_{NT} are the number of pixels for viable tumor and necrotic tumor, respectively. Necrosis ratios estimated by the deep learning model with necrosis ratios estimated by pathologists from pathology reports were compared to evaluate if the segmentation model can reproduce manually assessed necrosis ratio by experts.

### Patient Stratification

On the basis of necrosis ratio calculated by the segmentation model, patients were stratified to predict patient outcome. OS and PFS outcome data were collected from patient charts, and Kaplan-Meier curves were plotted. Because reproducible estimation of necrosis ratio without any variability is now possible with the deep learning model, the well-known cutoff threshold at 90% was not only tried but also tuned the cutoff threshold with an interval of 10% to objectively investigate various cutoff thresholds specifically for this segmentation model and this data set. The log-rank test was performed to evaluate patient stratification.

### 2. Results

### Necrosis Ratio Assessment

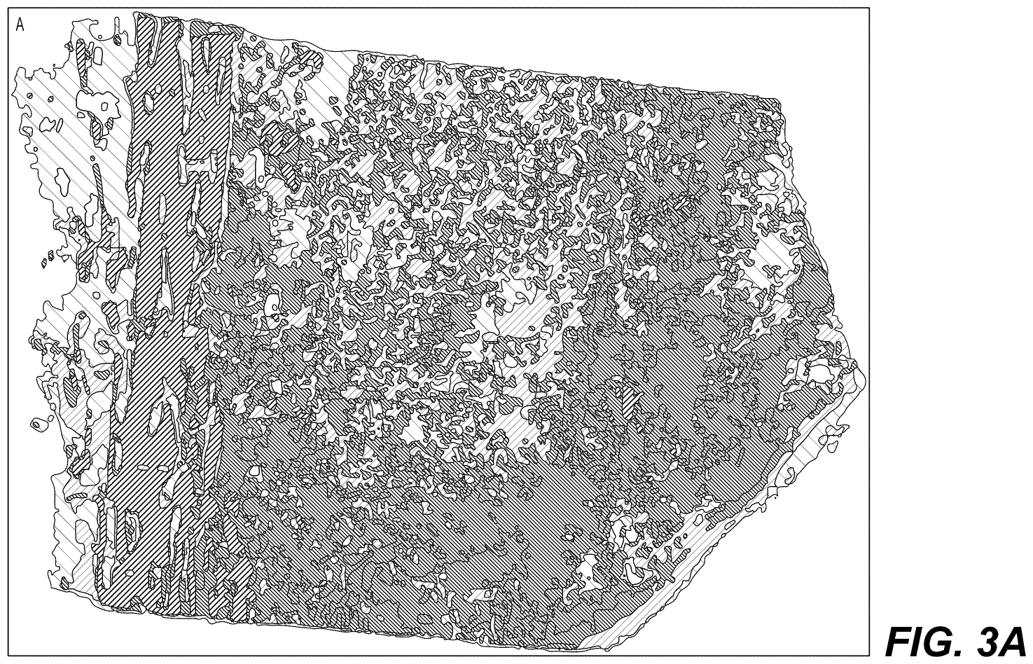

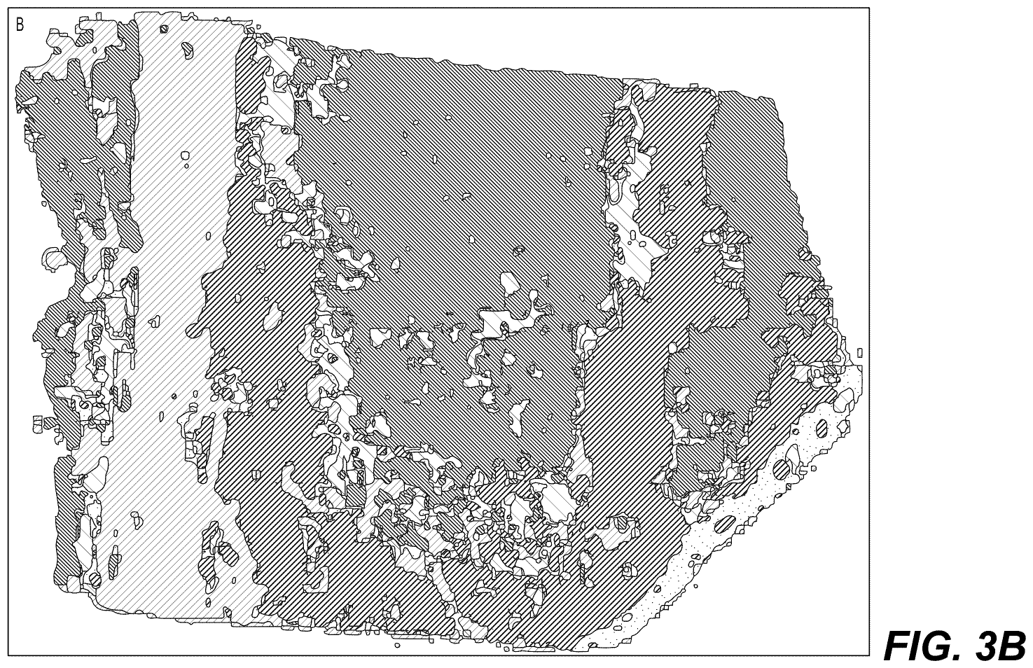

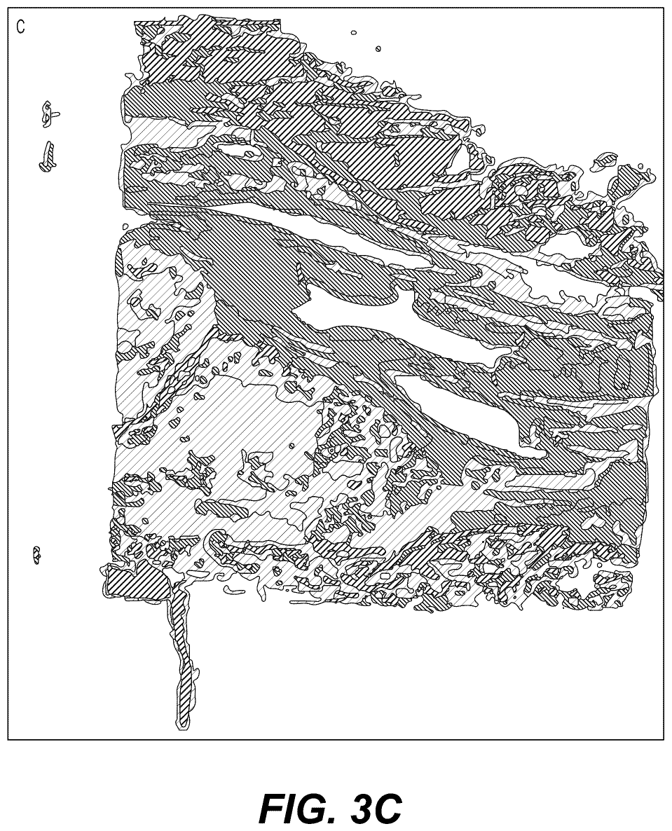

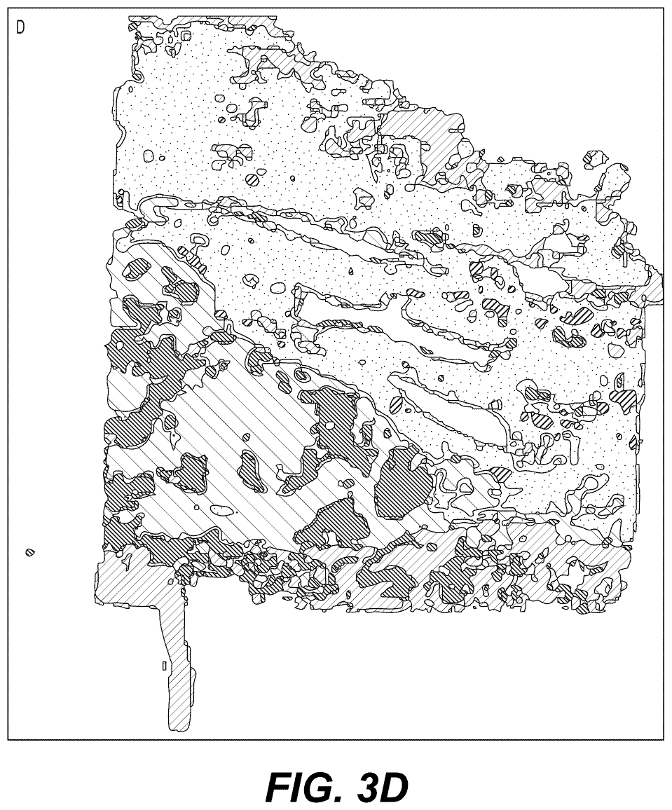

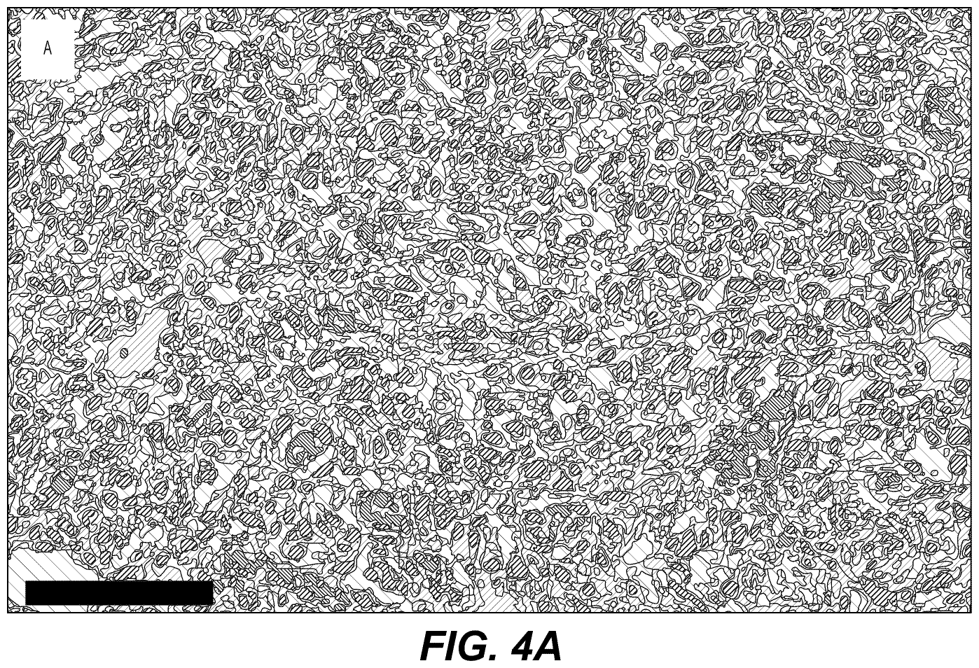

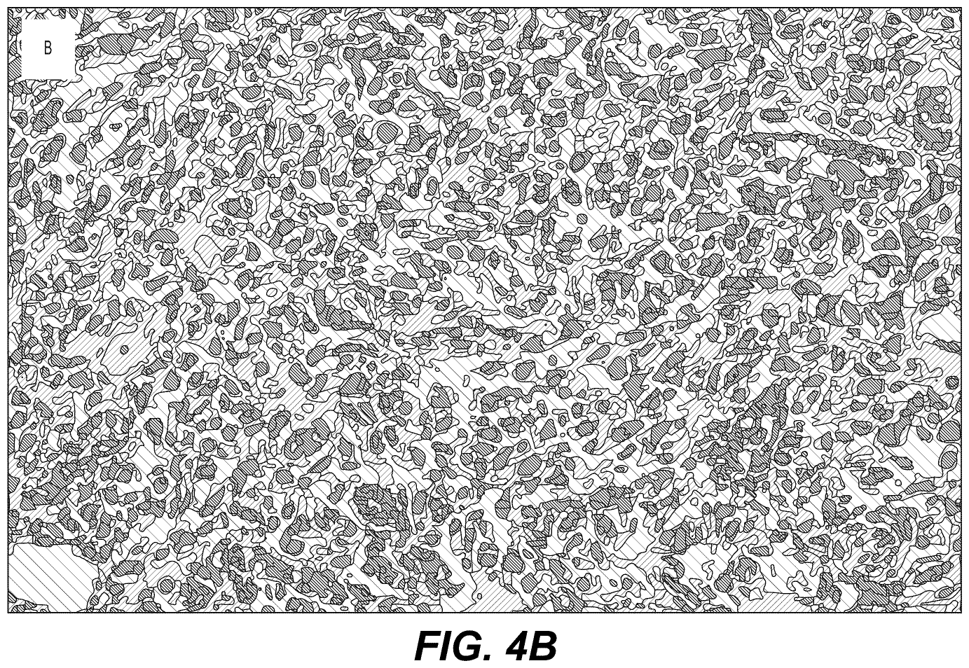

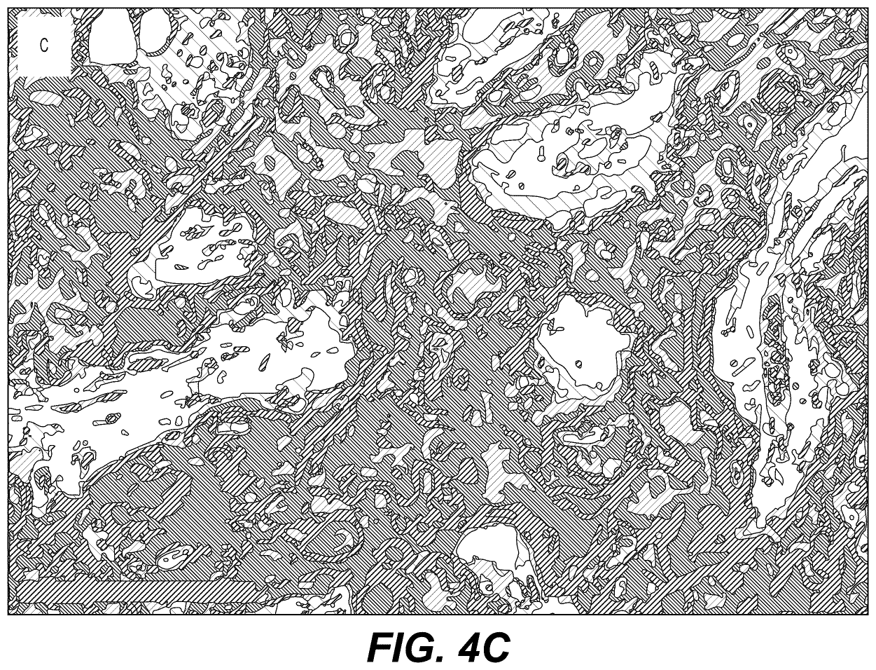

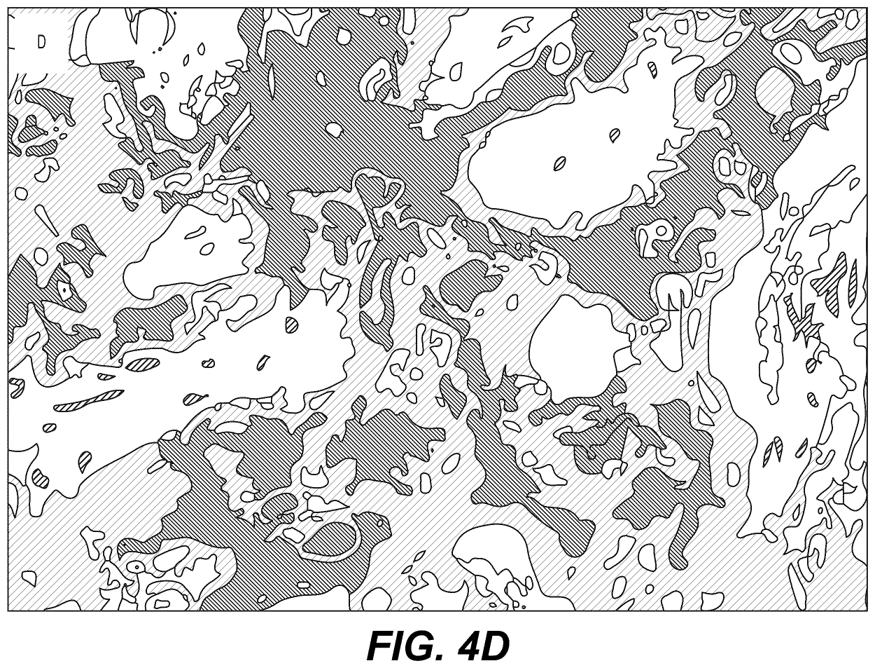

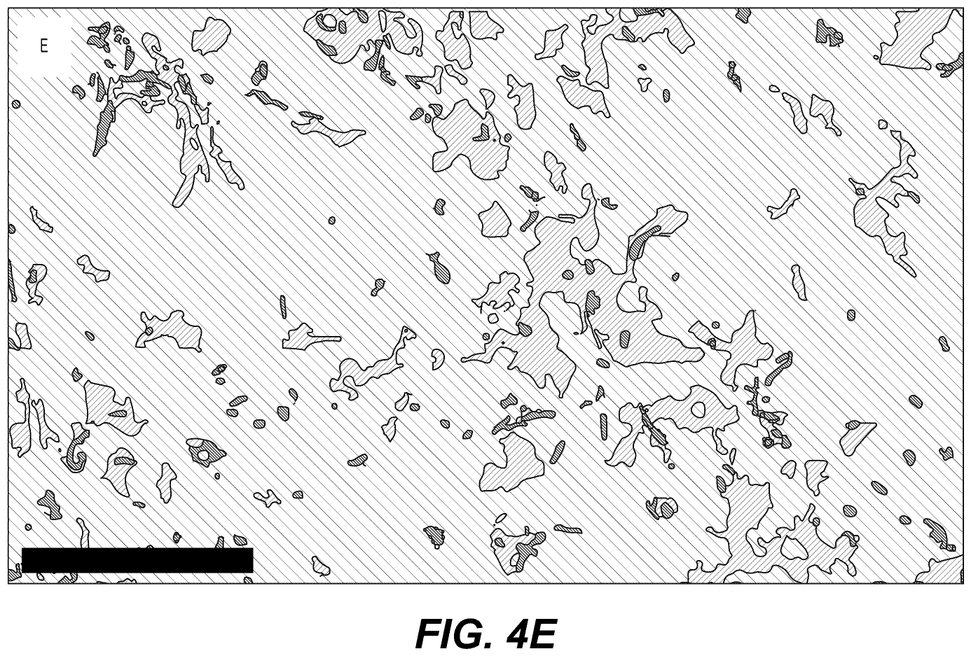

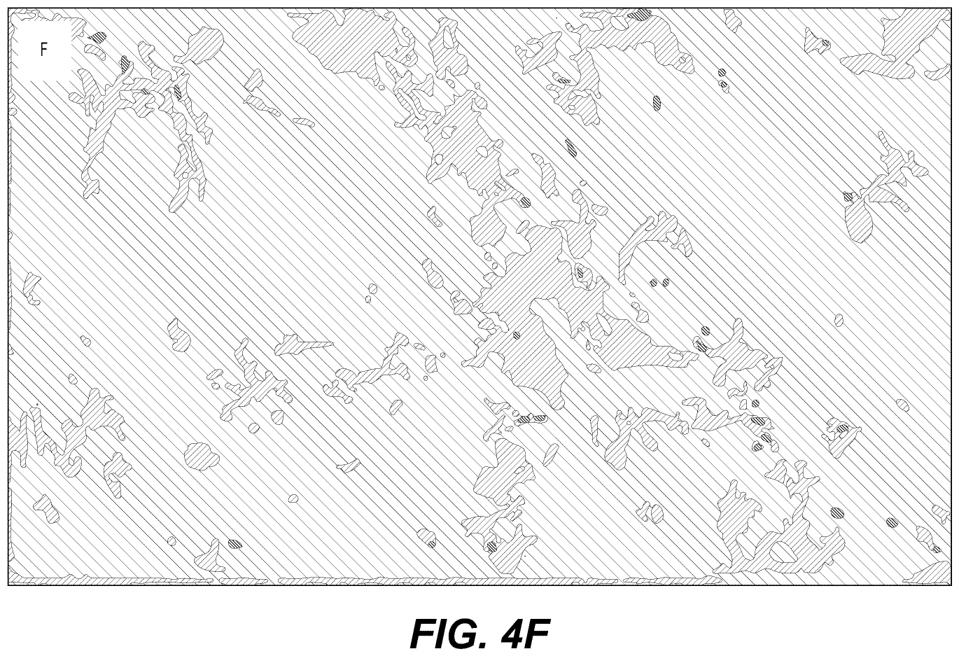

FIGS. 3 and 4 show multiclass segmentation predictions on WSIs and zoom-in images, respectively. By overlaying the multiclass segmentation predictions on testing WSIs using the Memorial Sloan Kettering Cancer Center slide viewer, the ability of the segmentation model to accurately segment the seven tissue subtypes was visually validated. The model was not able to accurately segment certain morphologic patterns, such as isolated viable tumor cells, chondroid foci, and densely sclerotic osteosarcoma, as shown in Supplemental Figure S1.

To quantitatively evaluate the segmentation model, necrosis ratio manually assessed by experts from pathology reports (denoted as r_{PR}) and necrosis ratio objectively assessed by the deep learning model (denoted as r_{DL}) were compared using absolute difference between them. The hypothesis was that the necrosis ratio of the deep learning model would be close to the necrosis ratio of manual assessment by experts. Therefore, absolute difference was used as a metric and was defined as |r_{PR}−r_{DL}|. Table 1 shows mean, median, and SD of absolute differences in various ranges of necrosis ratio. Mean and median absolute difference for cases whose necrosis ratio >90% were 4.44% and 2.95%, respectively. The scatterplot of the 80 testing cases is shown in Supplemental Figure S2. Three cases showed significant differences in necrosis ratio, where the pathologists' assessment described <50%, whereas the model predicted >90%. On rereview of the three cases, the consensus of pathologists' assessment of necrosis was 60% to 70%, which is still below the model's assessment of >90%. The contributing factors included isolated or small clusters of viable tumor cells, chondroid areas, and, in one case, densely sclerotic osteosarcoma with viable residual tumor cells. The model was further analyzed using outcome data of testing cases to evaluate if the deep learning model can be clinically used, as described in the next section.

### Outcome Prediction

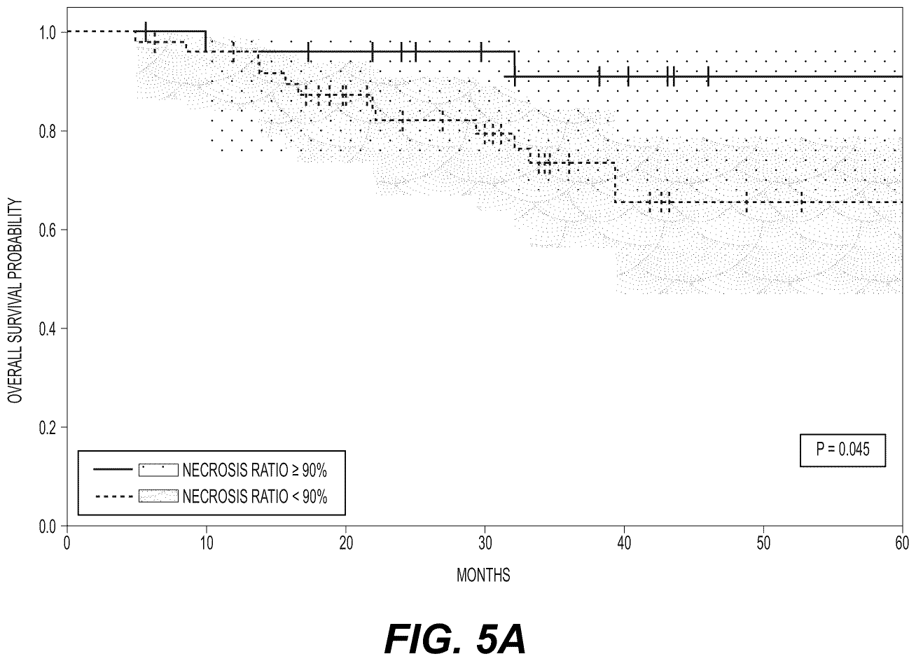

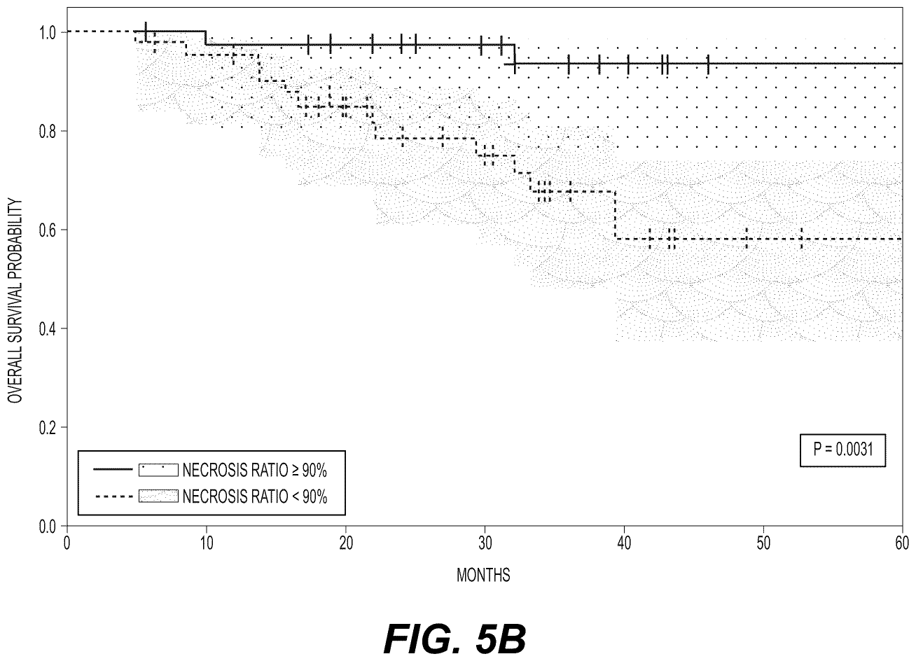

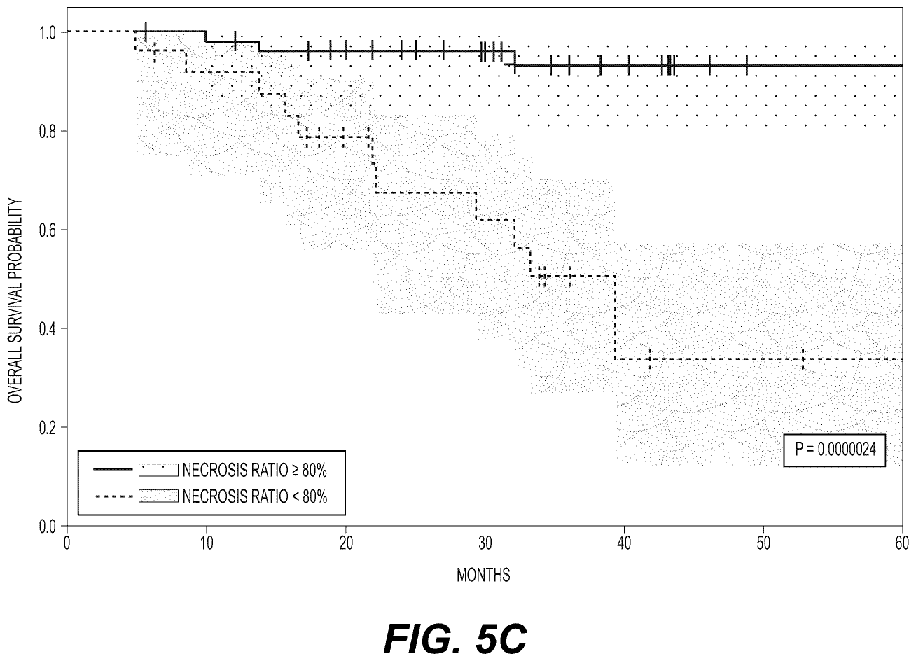

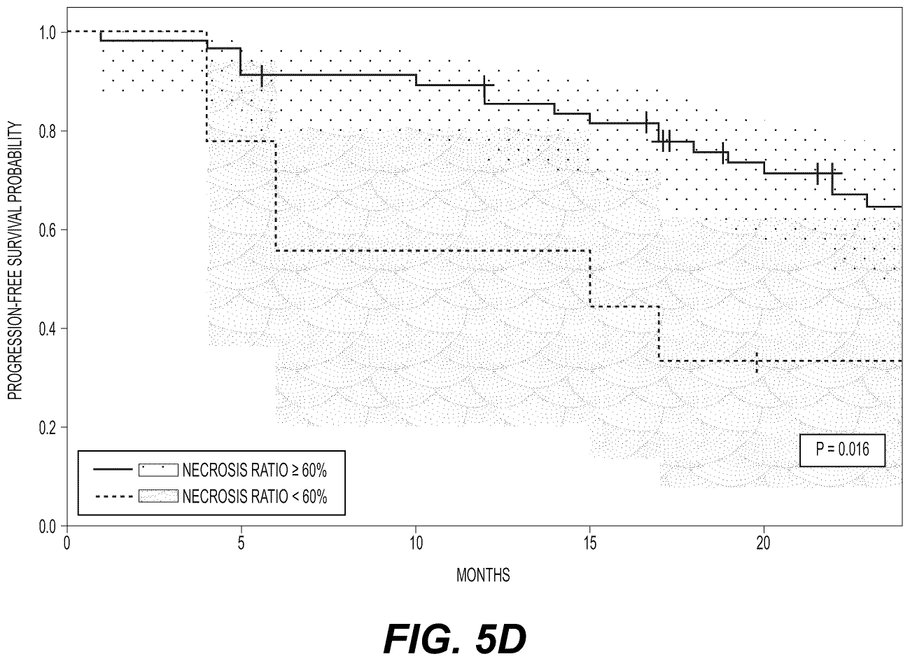

Kaplan-Meier curves were plotted, and the log-rank P values were calculated to evaluate outcome predictions, as shown in FIG. 5. On the basis of manual assessment from pathology reports at the conventional 90% cutoff threshold on 75 testing cases, P=0.045 was achieved for OS outcome. On the basis of automated assessment from the deep learning model at the 90% cutoff threshold, P=0.0031 was achieved, showing the deep learning model can successfully stratify patients for OS outcome. Because there is no variability caused by the deep learning model, an objective approach was proposed to investigate various cutoff thresholds, specifically for this segmentation model and this data set. With the interval of 10%, P=2.4×10⁻⁶ was achieved at the 80% cutoff threshold. Furthermore, PFS outcome data were predicted on 64 testing cases using the deep learning model, which achieved P=0.016 at the 60% cutoff threshold. The P values from various cutoff thresholds are shown in Supplemental Table S1.

### 3. Discussion

In this disclosure, a deep learning-based approach was developed to estimate case-level necrosis ratio from multiple H&E-stained osteosarcoma WSIs, where necrosis ratio is known to correlate with prognosis. Specifically, Deep Multi-Magnification Network was trained to objectively and reproducibly segment multiple tissue subtypes, including viable tumor and necrotic tumor, at a pixel level, to calculate necrosis ratio. The accuracy of necrosis ratio performed by the deep learning model was verified by comparing with manually assessed necrosis ratio from pathology reports. Furthermore, patients were stratified by OS and PFS based on necrosis ratio. Because of its objective manner, the cutoff threshold was tuned to stratify patients specifically for the trained model and the data set. The segmentation model achieved P=2.4×10⁻⁶ at the 80% cutoff threshold for OS and P=0.016 at the 60% cutoff threshold for PFS. This is the first study with the largest osteosarcoma cohort to compare manually assessed necrosis ratio from pathology reports to objectively assess necrosis ratio from the deep learning model and successfully stratify patients to predict OS and PFS based on objectively assessed necrosis ratio.

High intraobserver and interobserver variability of histologic subtypes of in situ and invasive cancer and necrosis percentage by manual microscopic assessment of H&E-stained sections has been observed in various cancer types, such as lung, breast, and colon. Although necrosis ratio from histologic slides is well proven as a prognostic factor in osteosarcoma, this visual estimation of necrosis remains subjective. Even with standardization of diagnostic criteria, reducing variability in necrosis ratio from ≥30 glass slides is challenging.

Deep learning with digitized histopathology images can be used as a tool to avoid this variability because deep learning models can objectively and consistently generate the same output given the same input. In this disclosure, the Deep Multi-Magnification Network was used to accurately segment viable tumor and necrotic tumor at the level of a pixel, the most basic element in an image. After seg-mentation of osteosarcoma WSIs, model performance was evaluated using manually assessed necrosis ratio from pathology reports and patient outcome data. For clinical relevance, necrosis ratios from the segmentation model were compared with necrosis ratios from pathology reports, taking into account the subjective nature of manual estimation of necrosis. Although necrosis ratio estimated by the segmentation model highly correlated with necrosis ratio manually assessed by experts in cases with high necrosis ratio, cases with necrosis ratio <50% generally had a higher absolute difference. This result is neither surprising nor unexpected. Manual assessment of necrosis ratio is known to be highly subjective. For example, it was shown that necrosis ratio assessed by six expert pathologists demonstrated an interclass correlation coefficient of 0.652 for 10 cases. In addition, high absolute differences within this range may be related to imprecise subjective estimation of low percentage of necrotic tumor, which is much below the cutoff threshold (90%) used to determine good or poor prognosis.

TABLE 1
Mean, Median, and SD of Absolute Differences on Various Ranges
Based on Necrosis Ratio from Pathology Reports, Denoted as r_{PR}

| Range | Mean | Median | SD | Cases, n |
|---|---|---|---|---|
| r_{PR} ≥ 90°/a | 4.44 | 2.95 | 4.17 | 26 |
| r_{PR} < 90°/a | 30.99 | 28.35 | 17.37 | 54 |
| 0°/a < r_{PR} < 100°/a | 22.37 | 17.9 | 19.09 | 80 |

For this reason, the necrosis ratio estimated by the model was used to stratify patients to predict OS and PFS outcome data. The log-rank test was used to verify better stratification by the segmentation model than by human experts. The cutoff threshold was tuned specifically for the segmentation model and the current data set because of its objective and reproducible manner. The outcome results validated the objectivity of the model to recognize necrosis patterns and to estimate percentage of treatment response. Previous studies have attempted to find the optimal cutoff threshold of necrosis ratio as a strong indicator of prognosis using manual assessment, but high intraobserver and interobserver variability has precluded effective conclusions. With a deep learning model, it would be possible to objectively and reproducibly select the optimal cutoff threshold stratifying patients with the lowest log-rank P value.

There are several qualifications to this study. The qualitative evaluation of segmentation predictions indicated that the segmentation model missed some viable tumors, such as isolated tumor cells, chondroid foci, and densely sclerotic osteosarcoma, potentially causing overestimation of necrosis ratio. The segmentation model was designed to segment at a tissue level, not at a cellular level. Although the model was able to segment regions with dense areas of viable tumor cells, it missed isolated viable tumor cells because of a lack of training by cell-level annotations. The estimation of necrosis ratio can be further improved by combining with a cell segmentation model that can detect isolated viable tumor cells. Chondroid foci and densely sclerotic osteosarcoma were underrepresented in the training set. By including more regions with rare patterns to the training set using Deep Interactive Learning or generating synthetic histology images with the rare patterns using generative adversarial networks, the model could be fine-tuned to accurately segment them. Artifacts caused during slide preparation (bone dust and stain precipitate) can lead to missegmentation, which is a common challenge in all digital and computational pathology. Training a more robust segmentation model by including artifacts in the training set would circumvent this issue. Lastly, this study was done with a data set from a single institution. For a more comprehensive study to improve segmentation and to select the optimal cutoff threshold, collecting a multi-institutional data set would be necessary.

In summary, the deep learning-based segmentation model was able to objectively and reproducibly estimate necrosis ratio from multiple osteosarcoma whole slide im-ages. The experimental results demonstrated high correlation between manually assessed necrosis ratio by pathologists and automatically calculated necrosis ratio by the segmentation model. This indicated that the segmentation model could successfully estimate osteosarcoma necrosis ratio from multiple slide images. Patients were stratified to predict overall survival and progression-free survival by additionally tuning the cutoff threshold in an objective manner. As intraobserver and interobserver variability is an intrinsic phenomenon in the manual and semiquantitative estimation of necrosis ratio, adopting deep learning-based models for a more objective assessment of necrosis ratio can pave the way for more prospective studies to assess treatment response and outcome in patients with osteosarcoma.

### 4. Supplemental Data

SUPPLEMENTAL TABLE
Log-rank p-values for overall survival (OS) and progression-
free survival (PFS) outcome data with various cutoff thresholds
for the segmentation model. Finding a cutoff threshold for
better stratification is possible for the deep learning-based
segmentation model because deep learning is objective and
reproducible. The minimum p-value for OS is achieved at the
80% cutoff threshold and the minimum p-value for PFS is achieved
at the 60% cutoff threshold, highlighted in bold.

| Cutoff | Overall | Progression-Free |
|---|---|---|
| Thresholds | Survival | Survival |
| 90% | 3.1 × 10⁻³ | 0.043 |
| 80% | 2.4 × 10⁻⁶ | 0.078 |
| 70% | 1.2 × 10⁻⁵ | 0.026 |
| 60% | 5.1 × 10⁻⁵ | 0.016 |
| 50% | 1.1 × 10⁻² | 0.086 |

B. Systems and Methods for Determining Predicted Outcomes of Subjects with Cancer from Biomedical Images

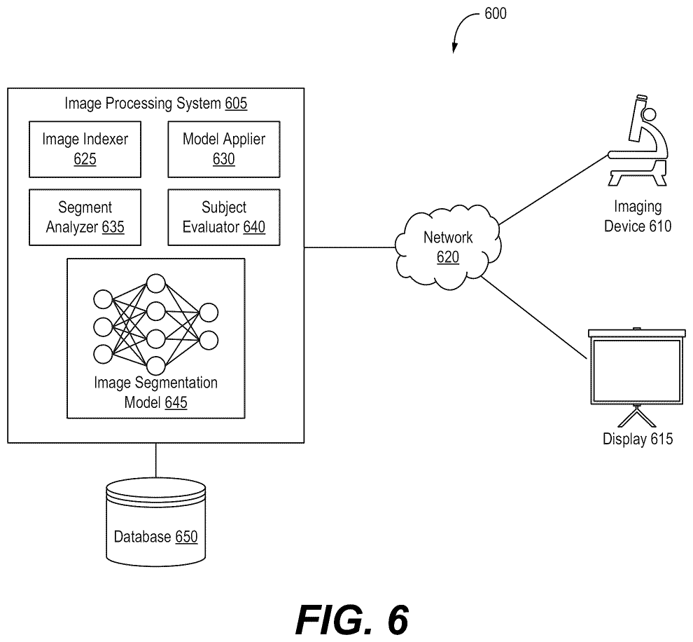

Referring now to FIG. 6, depicted is a block diagram of a system 600 for determining predicted outcomes of subjects with cancer from biomedical images. In overview, the system 600 may include at least one image processing system 605, at least one imaging device 610, and at least one display 615, among others, communicatively coupled with one another via at least one network 620. The image processing system 605 may include at least one image indexer 625, at least one model applier 630, at least one segment analyzer 635, at least one subject evaluator 640, at least one image segmentation model 645, and at least one database 650, among others. Each of the components in the system 100 as detailed herein may be implemented using hardware (e.g., one or more processors coupled with memory), or a combination of hardware and software as detailed herein in Section C. Each of the components in the system 100 may implement or execute the functionalities detailed herein, such as those described in Section A.

In further detail, the image processing system 605 may (sometimes herein generally referred to as a computing system or a server) be any computing device comprising one or more processors coupled with memory and software and capable of performing the various processes and tasks described herein. The image processing system 605 may be in communication with the imaging device 610 and display 615, and other devices, via the network 620. The image processing system 605 may be situated, located, or otherwise associated with at least one server group. The server group may correspond to a data center, a branch office, or a site at which one or more servers corresponding to the image processing system 605 is situated.

Within the image processing system 605, the image indexer 625 may retrieve, identify, or receive biomedical images from the imaging device 610 to be processed at the image processing system 605. The model applier 630 may apply biomedical images to the image segmentation model 645 to identify segments from the images. The image segmentation model 645 may have been initialized, trained, and established to identify the segments from the biomedical images using training data (e.g., in accordance with supervised learning techniques). The segment analyzer 635 may determine various characteristics associated with the segments identified from the segments. The subject evaluator 640 may generate information on the identified characteristics to provide for presentation on the display 615.

The image segmentation model 645 may be any type of machine learning algorithm or model to generate segmented images, such as a thresholding algorithm (e.g., Otsu's method), a clustering algorithm (e.g., k-means clustering), an edge detection algorithm (e.g., Canny edge detection), a region growing technique, a graph partitioning method (e.g., a Markov random field), and an artificial neural network (e.g., convolutional neural network architecture), among others. In general, the image segmentation model 645 may have at least one input and at least one output. The output and the input may be related via a set of weights. The input may be at least one an image (e.g., a biomedical image such as a whole slide image). The output may include at least one segmented image from the application of the image segmentation model 645 onto the input image in accordance with the set of weights.

In addition, the set of weights of the image segmentation model 645 may define corresponding parameters to be applied to the input image to generate the output image. In some embodiments, the set of weights may be arranged in one or more transform layers. Each layer may specify a combination or a sequence of application of the parameters to the input and resultant. The layers may be arranged in accordance with the machine learning algorithm or model for the image segmentation model 645. For example, the image segmentation model 645 may be a Deep Multi-Magnification Network (DMMN) as described herein in Section A. While discussed primarily in terms of artificial neural network architectures (e.g., DMMN), the image segmentation model 645 may be implemented using other architectures.

The imaging device 610 (sometimes herein generally referred to as an imaging device or an image acquirer) may be any device to acquire biomedical images of tissue samples from subjects at risk of or suffering from cancer. The subjects may be under guidance of clinician or hospital staff while scanned by the imaging device 610. The imaging device 610 may perform the scan in accordance with any number of imaging modalities, such as a whole slide imaging (WSI) for digital pathology (e.g., with a microscopy), X-ray scan, a computed tomography (CT) scan, a computed tomography laser mammography (CTLM), a magnetic resonance imaging (MRI) scan, a nuclear magnetic resonance (NMR) scan, an ultrasound imaging scan, a positron emission tomography (PET) scan, or a photoacoustic spectroscopy scan, among others.

The display 615 may be communicatively coupled with the image processing system 605 or any other computing device comprising one or more processors coupled with memory and software and capable of performing the various processes and tasks described herein. The display 615 may display, render, or otherwise present any information provided by the image processing system 605 or the images of subjects acquired via the imagining device 610. The information may be used by a clinician examining a subject to define an administration of a treatment to the subject.

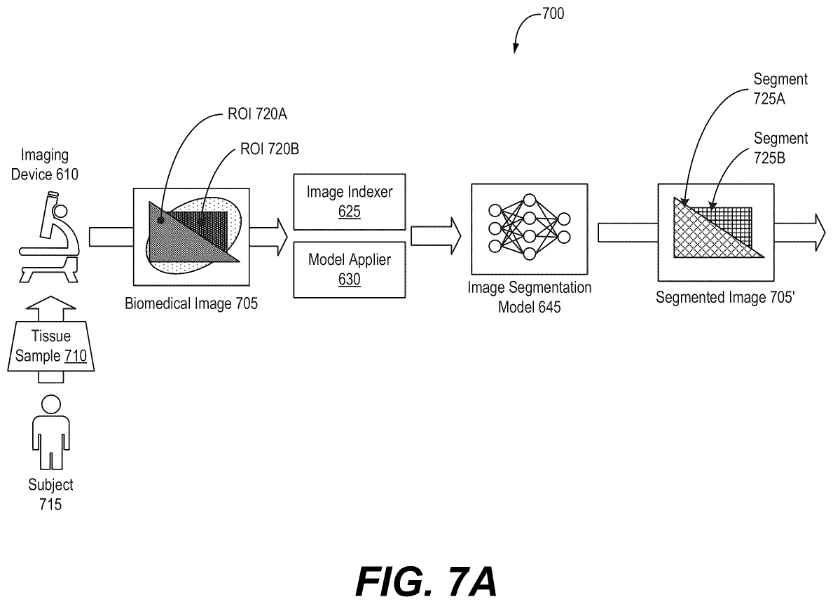

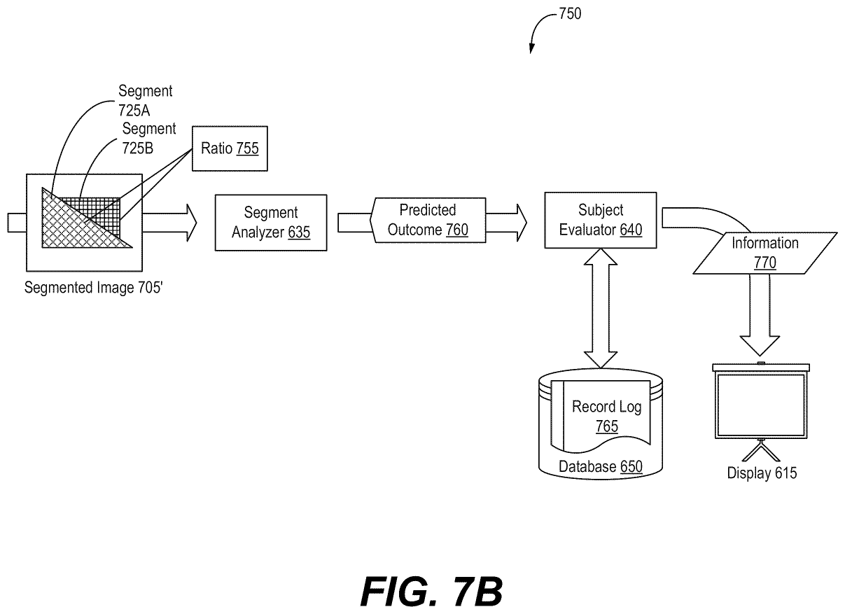

Referring now to FIG. 7A, depicted is a block diagram of a process 700 for segmenting biomedical images in the system 600 for determining predicted outcomes. The process 700 may include or correspond to operations performed in the system 600 for acquiring and segmenting biomedical images from subjects. Under the process 700, the imaging device 610 may output, produce, otherwise generate at least one biomedical image 705. The imaging device 610 may scan, obtain, or otherwise acquire the biomedical image 705 of a tissue sample 710 obtained from a subject 715. In some embodiments, the imaging device 610 generate a set of biomedical images 705. Each biomedical image 705 in the set may be of a respective tissue sample 710 from the subject 715. Each biomedical image 705 in the set may be acquired at a respective time instance. Upon acquisition, the imaging device 610 may send, transmit, or otherwise provide the biomedical image 705 to the image processing system 605. In some embodiments, imaging device 610 may store and maintain the biomedical image 705 on the database 650.

The subject 715 may be a human or an animal at risk of cancer or suffering from cancer. The cancer may include, for example, a bone cancer (e.g., osteosarcoma, chondrosarcoma, chordoma, or Ewing sarcoma), a lung cancer (e.g., non-small cell lung cancer (NSCLC) or Small cell lung cancer (SCLC)), a breast cancer (e.g., Ductal carcinoma in situ (DCIS), invasive ductal carcinoma (IDC), Lobular carcinoma in situ (LCIS), and Invasive lobular carcinoma (ILC)), and colon cancer (e.g., adenocarcinoma, Gastrointestinal carcinoid tumors, lymphomas), among others. The cancer may be present in the tissue sample 710 in the form of tumorous cells depicted in at least a portion of the biomedical image 705.

The tissue sample 710 may be taken, collected, or otherwise obtained from at least one anatomical site associated with the cancer within the subject 715. The tissue sample 710 can include tissue, bone, cartilage, or any other portion of the organ from the anatomical site. The anatomical site may be a primary site or a secondary (e.g., metastasized) site for the cancer. For example, when the subject 715 at risk of or suffering from thyroid cancer (e.g., papillary, follicular, medullary, or anaplastic), the clinician examining the subject 715 may collect the tissue samples 710 via biopsy from the thyroid of the subject 715. When multiple biomedical images 705 are acquired, the tissue samples 710 may be obtained from the same anatomical site (e.g., organ or other body party) associated with the cancer in the subject 715.

The tissue sample 710 may have one or more portions correspond to different tissue types (sometimes herein referred to as morphological classifications), such as: viable tumor; necrotic tumor; fibrotic; normal bone, tissue, or cartilage, and blank (or null), among others. The viable tumor may include or correspond to portions of the tissue sample 710 in which tumorous cells are active (e.g., actively reproducing). The necrotic tumor may include or correspond to portions of the tissues sample 710 that are dying (e.g., losing structural or cellular integrity). The fibrotic tissue may include or correspond to portions of the tissue sample 710 forming excessive fibrous tissue in response to a healing response to cancer or other injury. The normal bone, tissue, or cartilage may correspond or include healthy and functioning portions of the tissue sample 710.

In some embodiments, the tissue sample 710 may be stained, added to, or otherwise modified to facilitate the imaging of thereof. For example, the tissue section 808 may be stained with a hematoxylin and eosin (H&E), hemosiderin stain, a Sudan stain, a Schiff stain, a Congo red stain, a Gram stain, a Ziehl-Neelsen stain, a Auramine-rhodamine stain, a trichrome stain, a Silver stain, and Wright's Stain, among others. The stains may be used to differentiate among types of tissue within the tissue sample 710. For instance, the tissue sample 710 may be stained to differentiate the viable tumor and the necrotic tumor from a remainder of the tissue sample 710 (e.g., normal or blank). With the staining of the tissue sample 710, the imaging device 610 may acquire a whole slide image (WSI) of the tissue sample 710 placed on a slide to generate the biomedical image 705.

The biomedical image 705 may be acquired using any number of imaging modalities in accordance with lung cancer screening techniques. For example, the imaging modalities may include microscopy (e.g., in accordance with whole-slide imaging (WSI)), an X-ray scan, a computed tomography (CT) scan, a computed tomography laser mammography (CTLM), a magnetic resonance imaging (MRI) scan, a nuclear magnetic resonance (NMR) scan, an ultrasound imaging scan, a positron emission tomography (PET) scan, or a photoacoustic spectroscopy scan, among others. Although primarily discussed herein in terms of whole slide image (WSIs), other imaging modalities besides those listed above may be supported by the image processing system 105 for the biomedical image 705. The biomedical image 705 may be in the form of an image file (e.g., with a BMP, TIFF, LJPEG, or PNG, among others).

The biomedical image 705 may have at least one first region of interest (ROI) 720A (also referred herein as a structure of interest (SOI), a volume of interest (VOI), or feature of interest (FOI)) and at least one second ROI 720B. The first ROI 720A and the second ROI 720B may each correspond to at least one area, section, or portion of the biomedical image 705 correlated with a respective type of tissue in the tissue sample 710. The first ROI 720A may include the portion of the biomedical image 705 corresponding to the viable tumor in the tissue sample 715. The second ROI 720B may include the portion of the biomedical image 705 corresponding to the necrotic tumor in the tissue sample 715. The ROI 720A and 720B may each correspond to a contiguous portion (e.g., as depicted) or one or more non-contiguous portions within the biomedical image 705. The first ROI 720A and the second ROI 720B may be mutually exclusive (e.g., non-overlapping) within the biomedical image 705. The ROIs 720A and 720B may be unlabeled or not-yet-identified in the biomedical image 705.

The image indexer 625 executing on the image processing system 605 may retrieve, receive, or otherwise identify the biomedical image 705 of the tissue sample 710 from the subject 715. In some embodiments, the image indexer 625 may receive the biomedical image 705 acquired via the imaging device 610. In some embodiments, the image indexer 625 may access the database 650 to retrieve the biomedical image 705. In some embodiments, the image indexer 625 may identify the set of biomedical images 705 acquired over a corresponding set of time instances. Each biomedical image 705 in the set may be of a respective tissue sample 710 obtained from an anatomical site for the cancer in the subject 715 at a particular time instance. In some embodiments, the image indexer 625 may identify the set of biomedical images 705 acquired at a single time instance. In some embodiments, the image indexer 625 may determine or identify the time instance correspond to an acquisition of the biomedical image 705 from metadata associated with the biomedical image 705. For example, the metadata may identify or include a timestamp at which the biomedical image 705 is acquired.

The model applier 630 executing on the image processing system 605 may feed or apply the biomedical image 705 to the image segmentation model 645. As discussed above, the image segmentation model 645 may include the set of weights arranged in accordance with the model architecture to process the input biomedical image 705. In feeding, the model applier 630 may process the biomedical image 705 in accordance with the set of weights defined by the image segmentation model 645. For instance, the model applier 630 may apply the DMMN architecture detailed in Section A to the input biomedical image 705 to generate a segmentation of the input. From processing in accordance with the image segmentation model 645, the model applier 630 may produce, output, or otherwise generate at least one segmented image 705′. When the set of biomedical images 705 is identified, the model applier 630 may traverse through the set and apply each biomedical image 705 to the image segmentation model 645 to generate a corresponding segmented image 705′.

The segmented image 705′ may be a segmentation of the input biomedical image 705, and may have the same dimensions and format as the biomedical image 705. The segmented image 705′ may identify or include at least one first segment 725A and at least one second segment 725B. Each segment 725A and 725B may correspond to a respective morphological classification (e.g., one of the seven types of tissue discussed above) for the tissue sample 710 from which the biomedical image 705 is acquired. The first segment 725A may define, correspond to, or otherwise identify the first ROI 720A in the biomedical image 705 associated with the viable tumor in the tissue sample 710. The second segment 725B may define, correspond to, or otherwise identify the second ROI 720B in the biomedical image 705 associated with the necrotic tumor in the tissue sample 710. From the segmented image 705′ generated using the image segmentation model 645, the model applier 630 may identify or determine the first segment 725A and the second segment 725B.

In some embodiments, prior to the application of the newly acquired biomedical images (e.g., in the form of the biomedical image 705), the image segmentation model 645 may have been initialized, trained, and established (e.g., by the image processing system 605 or another computing system) using a training dataset. The training dataset may identify or include a set of examples. Each example may identify or include the biomedical image 705 and annotation for the biomedical image 705. The annotation may define, specify, or otherwise identify the first ROI 720A associated with the viable tumor in the tissue sample 710 and the second ROI 720B associated with the necrotic tumor in the tissue sample 710, within the biomedical image 705. The image segmentation model 645 may be initialized with the set of weights set to initial values (e.g., random values).

To train, the biomedical image 705 may be applied to the image segmentation model 645 to generate the segmented image 705′. The segmented image 705′ may identify or include the first segment 725A and the second segment 725B. A loss metric may be calculated, generated, or otherwise determined based on a comparison between the annotation for the biomedical image 705 and the segmented image 705′. The comparison may be between the first segment 725A as outputted by the image segmentation model 645 and the first ROI 720A as identified in the annotation and between the second segment 725B as outputted and the second ROI 720B as identified in the annotation. The loss metric may indicate a deviation between the output from the image segmentation model 645 and the expected output as identified in the annotation. The loss metric may be calculated in accordance with a root mean squared error, a relative root mean squared error, and a weighted cross entropy, among others. The weights of the image segmentation model 645 may be modified or updated using the loss metric in accordance with an objective function (e.g., stochastic gradient descent (SGD)). The image segmentation model 645 may be iteratively updated until convergence to complete the training.

Referring now to FIG. 7B, depicted is a block diagram of a process 750 for providing information on predicted outcomes in the system for determining predicted outcomes. The process 750 may include or correspond to operations to determine predicted outcomes using the segmentations identified from the biomedical images. Under the process 750, the segment analyzer 635 executing on the image processing system 605 may calculate, measure, or otherwise determine at least one ratio 755 (sometimes herein referred to as a necrosis ratio). The ratio 755 may be between a size of the first segment 725A associated with the viable tumor and a size of the second segment 725B associated with the necrotic tumor in the tissue sample 710. By extension, the ratio 755 may correspond to or identify an amount of viable tumor versus an amount of necrotic tumor in the tissue sample 710.

To determine, the segment analyzer 635 may determine a number of pixels corresponding to the first segment 725A and a number of pixels corresponding to the second segment 725B in the segmented image 705′. With the determination, the segment analyzer 635 may calculate the ratio 755 based on the number of pixels associated with the viable tumor and the number of pixels associated with the necrotic tumor in the tissue sample 710. For example, the segment analyzer 635 may determine the ratio 755 based on the number of pixels of the first segment 725A associated with the viable tumor, versus a sum of the number of pixels of the first segment 725A and the number of pixels associated with the necrotic tumor.

In some embodiments, with multiple segmented images 705′ derived from the set, the segment analyzer 635 may determine the size of the first segment 725A associated with the viable tumor and the size of the second segment 725B associated with the necrotic tumor for each segmented image 705′. The size of the first segment 725A and the size of the second segment 725B may each be the corresponding number of pixels in the segmented image 705′. With the determination, the segment analyzer 635 may calculate the ratio 755 between the size of the first segment 725A and the size of the second segment 725B. Using the ratios calculated over the set of segmented images 705′, the segment analyzer 635 may calculate, generate, or otherwise determine an aggregate ratio 755. The aggregate ratio 755 may, for example, be an average of the ratios calculated for the individual segmented images 705′.

With the determination of the ratio 755, the segment analyzer 635 may calculate, determine, or otherwise generate at least one value indicative of a predicted outcome 760 of the cancer in the subject 715. The predictive outcome 760 may define, correspond to, or otherwise identify a likelihood of the subject 715 to survive with the cancer at a given length of time (e.g., ranging from days to years relative to acquisition to the biomedical image 705). In some embodiments, the predictive outcome 760 may be an overall survival (OS) measure identifying a length of time during which the subject 715 with the cancer survives or is alive. In some embodiments, the predictive outcome 760 may be a progression free survival (PFS) measure identifying as length of time during which the cancer does not worsen or progress. The predictive outcome 760 may also identify a likelihood that the subject 715 is to have a positive response to treatment of the cancer. In some embodiments, the predictive outcome 760 may be a treatment measurement response identifying a likelihood that the cancer in the subject 715 is to respond (e.g., improve or treated) in response to treatment. The segment analyzer 635 may use a function to determine the value indicative of the predicted outcome 760 based on the ratio 755. The function may define a mapping or a correspondence between the ratio 755 and the values for the predictive outcome 760. When the biomedical images 705 used to generate the ratios 755 are acquired over the set of time instances, the segment analyzer 635 may generate the value indicative of the predicted outcome 760 at the respective time instance for each ratio 755.

The subject evaluator 640 executing on the image processing system 605 may determine, select, or otherwise classify the subject 715 into one of a set of risk stratification categories based on the value of the predicted outcome 760. The set of risk stratification categories may be used to differentiate subjects 715 in terms of impact or outcome from cancer. Each risk stratification category may correspond to a range of values for the predictive outcome 760 defined by at least one threshold. For example, the set of risk stratification categories may include a high-risk category and a low-risk category defined by a cutoff in the value for the predicted outcome 760. The high-risk category may include subjects 715 with low likelihood of OS, PFS, or treatment response. Conversely, the low-risk category may include subject 715 with high likelihood of OS, PFS, or treatment response.

To classify, the subject evaluator 640 may compare the value of the predicted outcome 760 with a threshold for each risk stratification category. The threshold may delineate, identify, or otherwise a specify a value (or a range of values) for the predicted outcome 760 at which to classify the subject 715 to the associated risk stratification category. When multiple predicted outcomes 760 are generated for the set of biomedical images 705 over the set of time instances, the subject evaluator 640 may perform the comparison for the value of each predicted outcome 760 at a respective time instance with the threshold. If the value of the predicted outcome 760 satisfies (e.g., greater than or equal to, or within) the threshold, the subject evaluator 640 may classify the subject 715 with the corresponding risk stratification category for the threshold. On the other hand, if the value of the predicted outcome 760 does not satisfy (e.g., less than, or outside) the threshold, the subject evaluator 640 may refrain from classifying the subject 715 with the corresponding risk stratification category for the threshold.

In some embodiments, the subject evaluator 640 may calculate, generate, or otherwise determine the threshold for each risk stratification category to compare against, using the values indicative of the predicted outcomes 760 determined from over a set of subjects 715. The threshold may correspond to a set percentage among the values indicative of predicted outcomes 760 from the overall set of subjects 715. For instance, the subject evaluator 640 may use the top 10% value of the predicted outcomes 760 as the threshold between the high-risk group and the low-risk group. In some embodiments, the subject evaluator 640 may use a distribution of the values indicative of the predicted outcomes 760 from the set of subjects 715 to determine the threshold. For example, the subject evaluator 640 may adjust, set, or optimize the threshold for statistical significance (e.g., P-value) of the predicted outcomes 760. In some embodiments, the threshold for each risk stratification category may be pre-defined or fixed.

With the classification, the subject evaluator 640 may store and maintain an association between the subject 715 (e.g., using an identifier) and the value indicative of the predictive outcome 760. The storage and maintenance of the association may use one or more data structures (e.g., arrays, matrixes, tables, linked lists, stacks, queues, trees, or heaps) on the database 650. In some embodiments, the subject evaluator 640 may store and maintain the association of the subject 715 with the biomedical image 705, the segment image 705′, the ratio 755 between the viable tumor and the necrosis tumor, the value indicative of the predicted outcome 760, and the classification of the subject 715 into one of the risk categories, among others.

In some embodiments, the subject evaluator 640 may keep track or maintain a measure of progression (or improvement) of the cancer in the subject 715 using the predicted outcomes 760 or the risk category over the set of time instances. To keep track, the subject evaluator 640 may store the values and classification for the subject 715 on a record log 765 maintained on the database 650. The record log 765 may identify the predicted outcome 760 or the classification (or both) for a particular subject 715 over the set of time instances (e.g., using the time stamp for each biomedical image 705). For each biomedical image 705 acquired at a respective time instance, the subject evaluator 640 may create or generate an entry identifying the predicted outcome 760 or classification to store onto the record log 765. Upon generation, the subject evaluator 640 may add or insert the entry onto the record log 765.

The subject evaluator 640 may send, transmit, or otherwise provide information 770 based on the association between the subject 715 and the value indicative of the predicted outcome 760 (or the classification into one of the risk categories). To provide, the subject evaluator 640 may create, produce, or otherwise generate the information 770 based on the association. The information 770 may identify or include, for example: the identifier for the subject 715, the biomedical image 705, the segmented image 705′, the segmentations 725A or 725B, the ratio 755, the value indicative of the predicted outcome 760, or the classification of the subject 715 into one of the risk categories, among others, or any combination thereof. The information 770 may be used (e.g., by the clinician examining the subject 715) to identify, specify, or otherwise define a treatment to administer to the cancer in the subject 715. With the generation, the subject evaluator 640 may provide the information 770 for presentation on the display 615.

The display 615 (or a computing device connected thereto) may display, render, or otherwise present the information 770 from the image processing system 605. The information 770 presented via the display 615 may be used by the clinician examining the subject 715 in defining a treatment to administer to the cancer in the subject 715. For example, when the subject 715 is suffering from osteosarcoma, the clinician may use the information 770 to decide which type of treatment (e.g., surgical removal, chemotherapy, radiation therapy, or targeted therapy) to apply and the parameters (e.g., locale or intensity) in administering the treatment. Upon receipt, the display 615 may render, display, or otherwise present the information 770. For instance, the display 615 may present the biomedical images 705 of an anatomical site associated with the cancer, adjacent to the segments 725A and 725B, the numerical value for the ratio 755, and the value indicative of the predicted outcome 760, among others.

In this manner, the image processing system 605 may use the image segmentation model 645 to apply on each biomedical image 705 to identify the segments 725A and 725B corresponding to viable and necrotic tumor tissues in the tissue sample 710 from the subject 715. The segments 725A and 725B may in turn be used by the image processing system 605 to calculate the necrosis ratio 755 in an objective and accurate manner, rather than a haphazard manner when examined manually and visually by a human clinician. Furthermore, the image processing system 605 may rely on the ratio 755 to determine the predicted outcome 760 to classify the subject 715 into the risk category to aid in defining the treatment for the subject 715. With higher accuracy and precision, the image processing system 605 can reduce the time spent in analyzing and evaluating the subject 715 with cancer, therefore allowing faster diagnosis and treatment. The image processing system 605 can also decrease the consumption of computing resource and network bandwidth that would have otherwise been spent using slide viewers to manually ascertain viable and necrosis tumors in tissue samples 715.

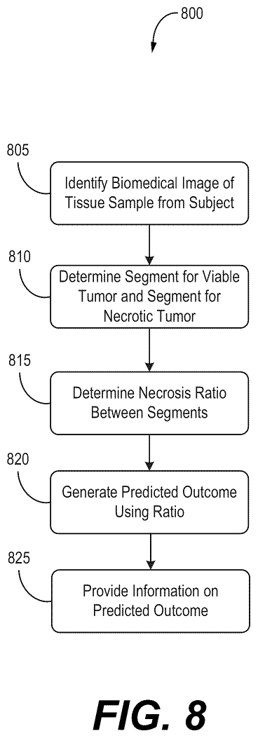

Referring now to FIG. 8, depicted is a flow diagram of a method 800 of determining predicted outcomes of subjects with cancer from biomedical images. The method 800 may be performed by or implemented using the system 600 described herein in conjunction with FIGS. 6-7B or the system 900 detailed herein in Section C. Under the method 800, a computing system (e.g., the image processing system 605) may identify a biomedical image (e.g., the biomedical image 715) of a tissue sample (e.g., the tissue sample 710) from a subject (e.g., the subject 715) (805). The computing system may determine a segment (e.g., the segment 725A) associated with a viable tumor and a segment (e.g., the segment 725B) associated with a necrotic tumor from the biomedical image (810). The computing system may determine a necrosis ratio (e.g., the ratio 730) between the segment associated with the viable tumor and the segment associated with the necrosis tumor (815). The computing system may generate a predicted outcome (e.g., the predicted outcome 760) using the ratio (820). The computing system may provide information (e.g., the information 770) on the predicted outcome (825).

### C. Computing and Network Environment

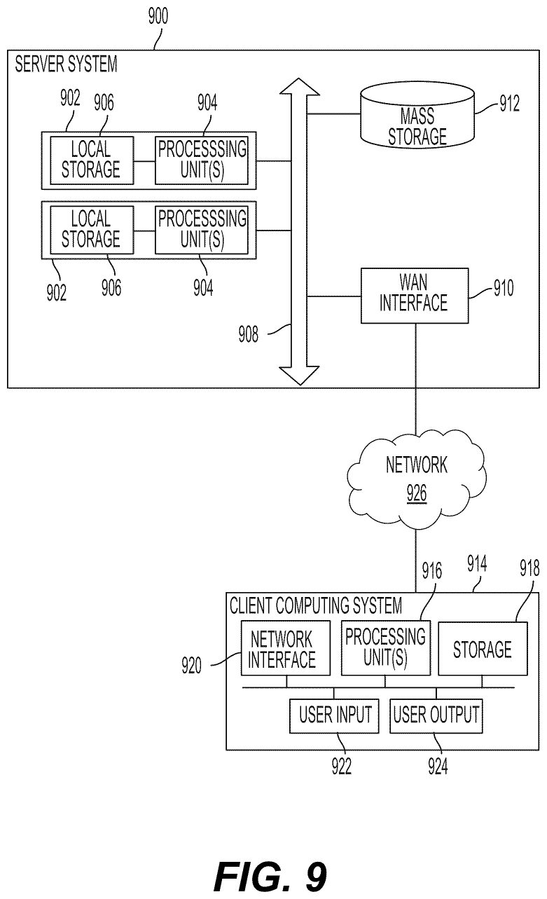

Various operations described herein can be implemented on computer systems. FIG. 9 shows a simplified block diagram of a representative server system 900, client computing system 914, and network 926 usable to implement certain embodiments of the present disclosure. In various embodiments, server system 900 or similar systems can implement services or servers described herein or portions thereof. Client computing system 914 or similar systems can implement clients described herein. The system 600 described herein can be similar to the server system 900. Server system 900 can have a modular design that incorporates a number of modules 902 (e.g., blades in a blade server embodiment); while two modules 902 are shown, any number can be provided. Each module 902 can include processing unit(s) 904 and local storage 906.

Processing unit(s) 904 can include a single processor, which can have one or more cores, or multiple processors. In some embodiments, processing unit(s) 904 can include a general-purpose primary processor as well as one or more special-purpose co-processors such as graphics processors, digital signal processors, or the like. In some embodiments, some or all processing units 904 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 904 can execute instructions stored in local storage 906. Any type of processors in any combination can be included in processing unit(s) 904.

Local storage 906 can include volatile storage media (e.g., DRAM, SRAM, SDRAM, or the like) and/or non-volatile storage media (e.g., magnetic or optical disk, flash memory, or the like). Storage media incorporated in local storage 906 can be fixed, removable or upgradeable as desired. Local storage 906 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 904 need at runtime. The ROM can store static data and instructions that are needed by processing unit(s) 904. The permanent storage device can be a non-volatile read-and-write memory device that can store instructions and data even when module 902 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 906 can store one or more software programs to be executed by processing unit(s) 904, such as an operating system and/or programs implementing various server functions such as functions of the system 600 of FIG. 6 or any other system described herein, or any other server(s) associated with system 600 or any other system described herein.

“Software” refers generally to sequences of instructions that, when executed by processing unit(s) 904 cause server system 900 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 904. Software can be implemented as a single program or a collection of separate programs or program modules that interact as desired. From local storage 906 (or non-local storage described below), processing unit(s) 904 can retrieve program instructions to execute and data to process in order to execute various operations described above.

In some server systems 900, multiple modules 902 can be interconnected via a bus or other interconnect 908, forming a local area network that supports communication between modules 902 and other components of server system 900. Interconnect 908 can be implemented using various technologies including server racks, hubs, routers, etc.

A wide area network (WAN) interface 910 can provide data communication capability between the local area network (interconnect 908) and the network 926, such as the Internet. Technologies can be used, including wired (e.g., Ethernet, IEEE 902.3 standards) and/or wireless technologies (e.g., Wi-Fi, IEEE 902.11 standards).

In some embodiments, local storage 906 is intended to provide working memory for processing unit(s) 904, providing fast access to programs and/or data to be processed while reducing traffic on interconnect 908. Storage for larger quantities of data can be provided on the local area network by one or more mass storage subsystems 912 that can be connected to interconnect 908. Mass storage subsystem 912 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 912. In some embodiments, additional data storage resources may be accessible via WAN interface 910 (potentially with increased latency).

Server system 900 can operate in response to requests received via WAN interface 910. For example, one of modules 902 can implement a supervisory function and assign discrete tasks to other modules 902 in response to received requests. Work allocation techniques can be used. As requests are processed, results can be returned to the requester via WAN interface 910. Such operation can generally be automated. Further, in some embodiments, WAN interface 910 can connect multiple server systems 900 to each other, providing scalable systems capable of managing high volumes of activity. Other techniques for managing server systems and server farms (collections of server systems that cooperate) can be used, including dynamic resource allocation and reallocation.

Server system 900 can interact with various user-owned or user-operated devices via a wide-area network such as the Internet. An example of a user-operated device is shown in FIG. 9 as client computing system 914. Client computing system 914 can be implemented, for example, as a consumer device such as a smartphone, other mobile phone, tablet computer, wearable computing device (e.g., smart watch, eyeglasses), desktop computer, laptop computer, and so on.

For example, client computing system 914 can communicate via WAN interface 910. Client computing system 914 can include computer components such as processing unit(s) 916, storage device 918, network interface 920, user input device 922, and user output device 924. Client computing system 914 can be a computing device implemented in a variety of form factors, such as a desktop computer, laptop computer, tablet computer, smartphone, other mobile computing device, wearable computing device, or the like.

Processing unit(s) 916 and storage device 918 can be similar to processing unit(s) 904 and local storage 906 described above. Suitable devices can be selected based on the demands to be placed on client computing system 914; for example, client computing system 914 can be implemented as a “thin” client with limited processing capability or as a high-powered computing device. Client computing system 914 can be provisioned with program code executable by processing unit(s) 916 to enable various interactions with server system 900.

Network interface 920 can provide a connection to the network 926, such as a wide area network (e.g., the Internet) to which WAN interface 910 of server system 900 is also connected. In various embodiments, network interface 920 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 922 can include any device (or devices) via which a user can provide signals to client computing system 914; client computing system 914 can interpret the signals as indicative of particular user requests or information. In various embodiments, user input device 922 can include any or all of a keyboard, touch pad, touch screen, mouse or other pointing device, scroll wheel, click wheel, dial, button, switch, keypad, microphone, and so on.

User output device 924 can include any device via which client computing system 914 can provide information to a user. For example, user output device 924 can include a display-to-display images generated by or delivered to client computing system 914. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 924 can be provided in addition to or instead of a display. Examples include indicator lights, speakers, tactile “display” devices, printers, and so on.

Some embodiments include electronic components, such as microprocessors, storage and memory that store computer program instructions in a computer-readable storage medium. Many of the features described in this specification can be implemented as processes that are specified as a set of program instructions encoded on a computer-readable storage medium. When these program instructions are executed by one or more processing units, they cause the processing unit(s) to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 904 and 916 can provide various functionality for server system 900 and client computing system 914, including any of the functionality described herein as being performed by a server or client, or other functionality.

It will be appreciated that server system 900 and client computing system 914 are illustrative and that variations and modifications are possible. Computer systems used in connection with embodiments of the present disclosure can have other capabilities not specifically described here. Further, while server system 900 and client computing system 914 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies including but not limited to the specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer-readable storage media; suitable media include magnetic disk or tape, optical storage media such as compact disk (CD) or DVD (digital versatile disk), flash memory, and other non-transitory media. Computer-readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

1. A method of determining predicted outcomes of subjects with cancer from biomedical images, comprising:
identifying, by a computing system, a biomedical image of a tissue sample from a subject with cancer, the biomedical image having (i) a first region of interest (ROI) corresponding to viable tumor in the tissue sample and (ii) a second ROI corresponding to necrotic tumor in the tissue sample;
applying, by the computing system, a machine learning model to the biomedical image to determine (i) a first segment identifying the first ROI in the biomedical image and (ii) a second segment identifying the second ROI in the biomedical image;
determining, by the computing system, a ratio between a first size of the first segment associated with the viable tumor and a second size of the second segment associated with the necrotic tumor;
generating, by the computing system, a value indicative of a predicted outcome of the cancer in the subject using the ratio; and
storing, by the computing system, using one or more data structures, an association between the subject and the value indicative of the predicted outcome.

2. The method of claim 1, further comprising:
classifying, by the computing system, the subject into a risk stratification category of a plurality of risk stratification categories based on a comparison of the value with a threshold, and
maintaining, by the computing system, a measure of progression of the cancer in the subject using the risk stratification category of the subject over a plurality of time instances.

3. The method of claim 2, further comprising determining, by the computing system, the threshold to compare against, based on a plurality of values each indicative of predicted outcome determined for a respective subject of a plurality of subjects.

4. The method of claim 1, wherein generating the value further comprises generating the value indicating at least one of an overall survival or a progression-free survival of the subject, based on the ratio of the first size of the first segment associated with the viable tumor and the second size of the second segment associated with the necrotic tumor.

5. The method of claim 1, wherein identifying the biomedical image further comprises receiving a corresponding plurality of biomedical images of a respective plurality of tissue samples obtained from an anatomical site for the cancer of the subject over a corresponding plurality of time instances, and
wherein generating the value further comprises generating the value indicative of the predicted outcome of the cancer for the subject at a respective time instance of the plurality of time instances at which the tissue sample was obtained.

6. The method of claim 1, wherein identifying the biomedical image further comprises receiving, via an imaging acquirer, a plurality of biomedical images each corresponding to a whole slide image (WSI) of a respective tissue sample stained to differentiate the viable tumor and the necrotic tumor from a remainder of the tissue sample, and
wherein determining the ratio further comprises determining the ratio between (i) a respective first size of the first segment associated with the viable tumor and (ii) a respective second size of the second segment associated with the necrotic tumor determined from each of the plurality of biomedical images.

7. The method of claim 1, wherein applying the machine learning model further comprises applying the machine learning model to the biomedical image to determine a plurality of segments, each of the plurality of segments corresponding to a respective morphological classification of a plurality of morphological classifications for the tissue sample.

8. The method of claim 1, wherein the first size identifies a first number of pixels of the first segment associated with the viable tumor, and wherein the second size identifies a second number of pixels of the second segment associated with the necrotic tumor.

9. The method of claim 1, wherein the machine learning model is established using a training dataset comprising a plurality of examples, each of the plurality of examples identifying (i) a respective second biomedical image of a second tissue sample having (a) a third ROI corresponding to viable tumor in the second tissue sample and (b) a fourth ROI corresponding to necrotic tumor in the second tissue and (ii) an annotation identifying the third ROI and the fourth ROI in the respective second biomedical image.

10. The method of claim 1, further comprising providing, by the computing system, information to define a treatment to administer to the cancer in the subject based on the association between the value and the subject, wherein the cancer includes one of bone cancer, lung cancer, breast cancer, or colon cancer.

11. A system for determining predicted outcomes of subjects with cancer from biomedical images, comprising:
a computing system having one or more processors coupled with memory, configured to:
identify a biomedical image of a tissue sample from a subject with cancer, the biomedical image having (i) a first region of interest (ROI) corresponding to viable tumor in the tissue sample and (ii) a second ROI corresponding to necrotic tumor in the tissue sample;
apply a machine learning model to the biomedical image to determine (i) a first segment identifying the first ROI in the biomedical image and (ii) a second segment identifying the second ROI in the biomedical image;
determine a ratio between a first size of the first segment associated with the viable tumor and a second size of the second segment associated with the necrotic tumor;
generate a value indicative of a predicted outcome of the cancer in the subject using the ratio; and
store, using one or more data structures, an association between the subject and the value indicative of the predicted outcome.

12. The system of claim 11, wherein the computing system is further configured to:
classify the subject into a risk stratification category of a plurality of risk stratification categories based on a comparison of the value with a threshold, and
maintain a measure of progression of the cancer in the subject using the risk stratification category of the subject over a plurality of time instances.

13. The system of claim 12, wherein the computing system is further configured to determine the threshold to compare against, based on a plurality of values each indicative of predicted outcome determined for a respective subject of a plurality of subjects.

14. The system of claim 11, wherein the computing system is further configured to generate the value indicating at least one of an overall survival or a progression-free survival of the subject, based on the ratio of the first size of the first segment associated with the viable tumor and the second size of the second segment associated with the necrotic tumor.

15. The system of claim 11, wherein the computing system is further configured to:
receive a corresponding plurality of biomedical images of a respective plurality of tissue samples obtained from an anatomical site for the cancer of the subject over a corresponding plurality of time instances, and
generate the value indicative of the predicted outcome of the cancer for the subject at a respective time instance of the plurality of time instances at which the tissue sample was obtained.

16. The system of claim 11, wherein the computing system is further configured to:
receive, via an imaging acquirer, a plurality of biomedical images each corresponding to a whole slide image (WSI) of a respective tissue sample stained to differentiate the viable tumor and the necrotic tumor from a remainder of the tissue sample, and
determine the ratio between (i) a respective first size of the first segment associated with the viable tumor and (ii) a respective second size of the second segment associated with the necrotic tumor determined from each of the plurality of biomedical images.

17. The system of claim 11, wherein the computing system is further configured to apply the machine learning model to the biomedical image to determine a plurality of segments, each of the plurality of segments corresponding to a respective morphological classification of a plurality of morphological classifications for the tissue sample.

18. The system of claim 11, wherein the first size identifies a first number of pixels of the first segment associated with the viable tumor, and wherein the second size identifies a second number of pixels of the second segment associated with the necrotic tumor.

19. The system of claim 11, wherein the machine learning model is established using a training dataset comprising a plurality of examples, each of the plurality of examples identifying (i) a respective second biomedical image of a second tissue sample having (a) a third ROI corresponding to viable tumor in the second tissue sample and (b) a fourth ROI corresponding to necrotic tumor in the second tissue and (ii) an annotation identifying the third ROI and the fourth ROI in the respective second biomedical image.

20. The system of claim 11, wherein the computing system is further configured to provide information to define a treatment to administer to the cancer in the subject based on the association between the value and the subject, wherein the cancer includes one of bone cancer, lung cancer, breast cancer, or colon cancer.
